(19) United States
(12) Patent Application Publication
(10) Pub. No.: US 2003/0134316 A1
(43) Pub. Date: Jul. 17, 2003

(54) Method of stirring reaction solutions

(76) Inventors: Hideo Tashiro, Wako-shi (JP); Tokuji Kitsunai, Wako-shi (JP); Yasumitsu Kondoh, Wako-shi (JP); Chikara Koike, Wako-shi (JP)

Correspondence Address: BIRCH STEWART KOLASCH & BIRCH, PO BOX 747, FALLS CHURCH, VA, 22040-0747, US

(21) Appl. No.: 10/321,677

(22) Filed: Dec. 18, 2002

(30) Foreign Application Priority Data
Dec. 18, 2001 (JP) 2001-384916
Nov. 22, 2002 (JP) 2002-339344

(51) Int. Cl. C12Q 1/68; B01F 13/08; C12M 1/34

(52) U.S. Cl. 435/6; 435/287.2; 366/274

(57) ABSTRACT

A method of stirring a reaction solution in a micro reaction vessel by imparting magnetic field fluctuation from the exterior of said reaction vessel to magnetic beads contained in said reaction solution. The reaction solution in a micro reaction vessel can be efficiently stirred.

### TECHNICAL FIELD

The present invention relates to a method of stirring reaction solutions in micro reaction vessels.

### TECHNICAL BACKGROUND

A number of companies supplying DNA chips and DNA microarrays have appeared. These products have thus become readily available and are expected to be widely employed in fields such as genetic diagnosis. DNA chips and DNA microarrays consist of several thousands to several tens of thousands of kinds of DNA fragments serving as indicators (probe DNA) densely arrayed on a glass slide, silicon substrate, or the like, which are hybridized by being immersed in or applied with a solution of DNA (target DNA) that is to be identified. Although automatic hybridization devices have begun to appear, hybridization is still widely manually conducted for reasons of stability, cost, and the like.

The hybridization of target DNA on a DNA chip or DNA microarray requires the placement of drops of target DNA-comprising sample in quantities of several microliters to several tens of microliters on the DNA chip or DNA microarray, covering with a cover glass, and maintaining this arrangement for several hours. Achieving reliable hybridization results requires that the target DNA be brought near to and placed in a state permitting hybridization with the probe DNA on the DNA chip or DNA microarray. However, due to the small quantities of solution, it is difficult to stir the solution. When stirring is not conducted, complete hybridization requires from 18 to 24 hours. Even in commercial hybridization devices having stirring functions, complete hybridization requires about four hours. Further, in commercial hybridization devices having stirring functions, from 100 to 400 microliters of sample are required. Still further, commercial hybridization devices have a drawback in that they are comprised of complex mechanisms and are thus expensive.

In manual hybridization method, it is sometimes impossible to obtain good, reproducible data due to failed hybridization. There is also a problem in the form of variation in results due to the individual conducting the hybridization. As DNA chips and DNA microarrays come into wider use, there is a need for a method of efficiently stirring the reaction solution in micro reaction vessels, such as when hybridizing target DNA in DNA chips and DNA microarrays.

Accordingly, the object of the present invention is to provide a method of efficiently stirring the reaction solutions in micro reaction vessels.

### SUMMARY OF THE INVENTION

The invention solving the above-stated problems is as follows:

(1) A method of stirring a reaction solution in a micro reaction vessel wherein a magnetic field fluctuation is imparted from the exterior of said reaction vessel to magnetic beads contained in said reaction solution.

(2) The method according to (1) wherein said magnetic field fluctuation is conducted by sequentially exciting multiple electromagnets or by displacing permanent magnets positioned outside the reaction vessel.

(3) The method according to (1) or (2) wherein the micro reaction vessel is a DNA chip or DNA microarray hybridization vessel.

(4) The method according to any of (1) to (3) wherein the thickness of the interior of the micro reaction vessel ranges from about 0.1 to 1 mm and the diameter of the magnetic beads ranges from about 0.1 to 20 percent of said thickness.

(5) The method according to any of (1) to (4) wherein the volume of said micro reaction vessel ranges from about 10 to 1,000 microliters.

(6) The method according to any of (1) to (5) wherein magnetic beads are employed that constitute from about 0.1 to 10 percent of the volume of the reaction solution.

### BRIEF DESCRIPTION OF THE DRAWINGS

FIG. 1 is a schematic diagram of a hybridization device in the implementation of the method of the present invention.

FIG. 2 is a conceptual drawing of stirring with the magnetic beads in the method of the present invention.

FIG. 3 is a schematic diagram of a hybridization device in which multiple micro reaction vessels are equipped for implementation of the present invention.

FIG. 4 gives the results of hybridization implemented in the embodiment.

### MODES OF IMPLEMENTING THE INVENTION

The present invention, a method of stirring a reaction solution in a micro reaction vessel, is characterized in that magnetic field fluctuation is imparted from the exterior of the reaction vessel to magnetic beads contained in the reaction solution.

The micro reaction vessel in the present invention refers to, for example, a DNA chip or DNA microarray hybridization vessel. However, the micro reaction vessel is not limited to hybridization vessels.

The micro reaction vessel may range in capacity from 10 to 1,000 microliters, preferably from 100 to 300 microliters. Further, in the case of hybridization vessels, the micro reaction vessel may comprise two opposing plates (for example, slide glass) and a spacer (for example, an O-ring) permitting the sealing of reaction solution between the two plates. In such cases, the thickness of the interior of the micro reaction vessel (corresponding to the thickness of the spacer member) may range from 0.1 to 1 mm, for example.

At least one of the two plates constituting the above-mentioned micro reaction vessel may be a DNA chip or DNA microarray on the surface of which DNA has been immobilized. Further, the reaction solution may be a hybridization solution comprising target DNA.

In the method of the present invention, a reaction solution and magnetic beads are sealed within the above-described micro reaction vessel and magnetic field fluctuation is imparted from the exterior of the reaction vessel to stir the reaction solution. From the perspectives of readily displacing the magnetic beads and efficiently stirring the reaction solution, the diameter of the magnetic beads suitably ranges from about 0.1 to 20 percent, preferably ranging from about 1 to 10 percent, of the thickness of the above-described micro reaction vessel. Specifically, the diameter of the magnetic beads ranges from about 0.001 to 0.1 mm. Magnetic beads of uniform diameter and magnetic beads of nonuniform diameter may be intentionally employed. The type of magnetic bead employed may be suitably determined based on the type of reaction.

However, from the perspective of avoiding unintended reactions with components of the reaction solution and components immobilized on the plates, the surface of the magnetic beads is desirably treated with a resin (for example, polypropylene) tending not to react with such components.

Further, from the perspective of readily imparting movement to the magnetic beads and efficiently stirring the reaction solution, the quantity of magnetic beads employed suitably falls within a range of from 0.1 to 20 volume percent, preferably within a range of from 1 to 10 volume percent, of the reaction solution.

The magnetic field fluctuation employed to move the magnetic beads may be applied by sequentially exciting multiple electromagnets or moving permanent magnets positioned outside the reaction vessel.

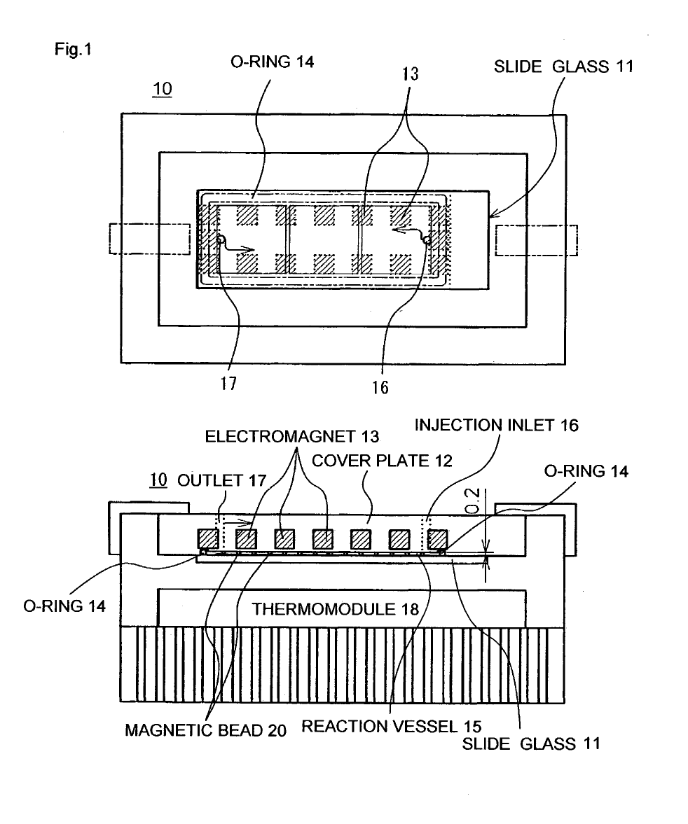

The case of stirring the reaction solution by moving magnetic beads within the reaction solution by imparting magnetic field fluctuation from the exterior of the reaction vessel will be described based on FIG. 1.

FIG. 1 is a schematic diagram of a hybridization device in the implementation of the method of the present invention. The upper diagram is a plan view and the lower diagram is a lateral view. Hybridization device 10 comprises a slide glass 11 (for example, a slide glass with a DNA array); a cover plate 12, in which at least one electromagnet 13 is embedded, positioned opposite slide glass 11; an O-ring 14, serving as a spacer, used to maintain a gap between glass slide 11 and cover plate 12; an injection inlet 16 into reaction vessel 15; an outlet 17, and a thermomodule 18. Reaction vessel 15 is comprised of slide glass 11, cover plate 12, and O-ring 14, measuring about 20×60 mm with a thickness of about 0.2 mm and a volume of about 250 microliters.

A prescribed volume (about 250 microliters) of reaction solution comprising magnetic beads 20 is injected through injection inlet 16 into reaction vessel 15. As shown in the upper diagram of FIG. 1, multiple electromagnets 13 are arranged (embedded) above and around slide glass 11 in cover plate 12. Once the reaction solution has been injected, multiple electromagnets 13 are sequentially excited. As that occurs, the magnetic beads move in the direction of cycling electromagnets 13. The movement (flow) of magnetic beads in the reaction solution causes the magnetic solution to rotate and be stirred.

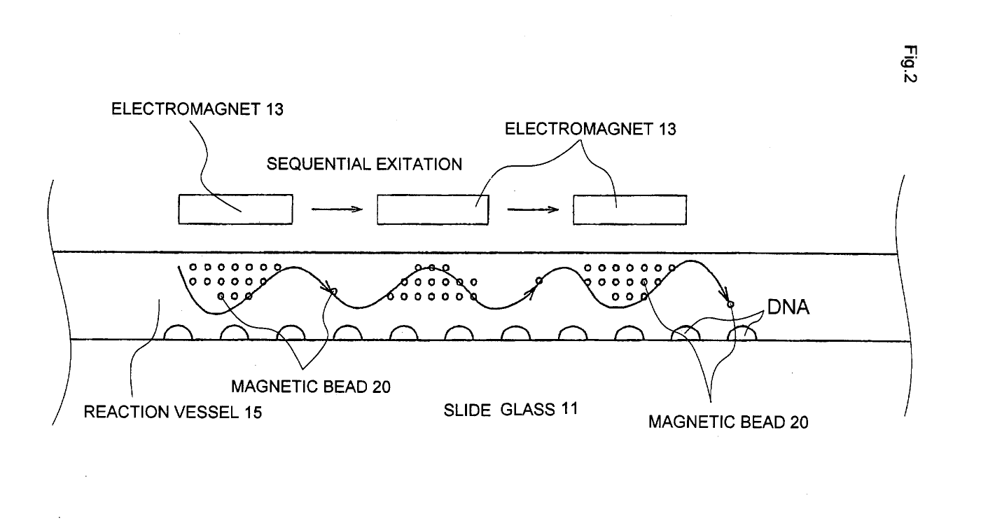

This state of magnetic beads in reaction vessel is shown in FIG. 2. In FIG. 2, electromagnets 13 are sequentially excited from left to right, magnetic beads 20 are attracted by the excited electromagnets, and as the excited electromagnets shift, magnetic beads 20 sequentially move from left to right.

In FIG. 1, multiple electromagnets 13 are arranged (embedded) in cover plate 12 to impart rotation to whatever is on slide glass 11. However, in addition to an arrangement imparting rotation, for example, the magnets may be arranged in cover plate 12 in a straight line in the longitudinal direction of slide glass 11 from one end to the other, or in a zigzag configuration. Further, in the above example, movement of the magnetic beads is imparted with electromagnets. However, in addition to electromagnets, permanent magnets or the like may also be employed.

During stirring of the reaction solution by causing the magnetic beads to move (flow), the temperature of the reaction solution may be adjusted with thermomodule 18 to a temperature suited to the reaction. In hybridization, the temperature of the reaction solution may be from room temperature to 90° C., for example. Further, the reaction time may be suitably determined based on the type of reaction. However, in the method of the present invention, since efficient stirring of even micro amounts of reaction solution is possible, the reaction time can be shortened.

Once the reaction has ended, the reaction solution is discharged through outlet 17 and the interior of the reaction vessel is suitably cleaned and dried. In the case of a DNA chip or DNA microarray, slide glass 11 can be took out and employed in hybridization detection operations (for example, fluorometric analysis to detect hybridized DNA). Further, the magnetic beads that are recovered with the reaction solution can be separated from the reaction solution, cleaned, dried, and reused.

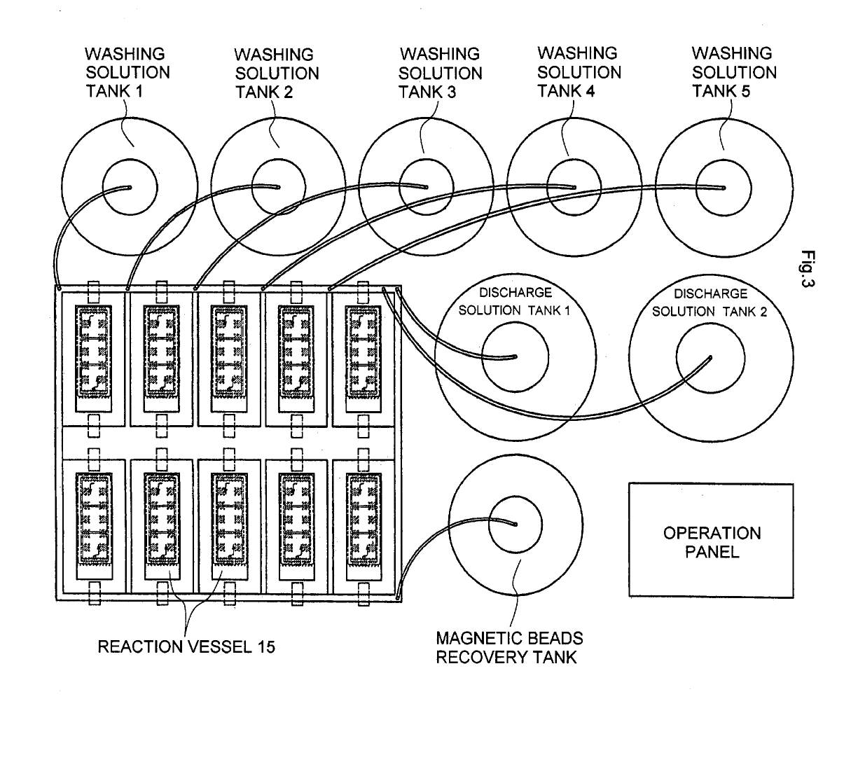

FIG. 1 shows a single reaction vessel. However, devices employing the method of the present invention may be configured as multiple devices equipped with multiple reaction vessels, units for supplying reaction solution and cleaning solution to the reaction vessels (reaction solution tanks, cleaning solution tanks, solution delivery pipes and pumps, and the like), and units for recovering discharged solution and magnetic beads (discharge solution tanks, magnetic bead recovery tanks, solution deliver pipes and pumps, and the like). See FIG. 3; the device shown in FIG. 3 is equipped with ten reaction vessels 15.

### Embodiment

The present invention is described in greater detail below through an embodiment.

Protocols

1. A slide glass (DNA microarray) stamped with the probes stated below was placed on hybridization cassettes such as that shown in FIG. 1 and heated to 65° C. Stamping of probe on the slide glass was performed in a manner yielding dots each of which had a diameter of about 100 to 150 micrometers, with 441 dots formed on each slide glass.

2. Following heating, the target solution (350 microliters) given below was poured onto the slide glass.

3. Heating (hybridization) was then conducted for 16 hours.

During heating in the embodiment of the present invention (with stirring), magnetic beads in the solutions were displaced by means of back and forth movement of permanent magnets in the upper portion of the hybridization cassette to stir the solutions. The stirring speed was set to 5 mm/s. In the comparative example (no stirring), the same procedure as in the embodiment was followed with the exception that the permanent magnets were not moved back and forth.

4. When 16 hours had elapsed, the slide glass was sequentially rinsed with 2×SSC, 1×SSC, and 0.2×SSC.

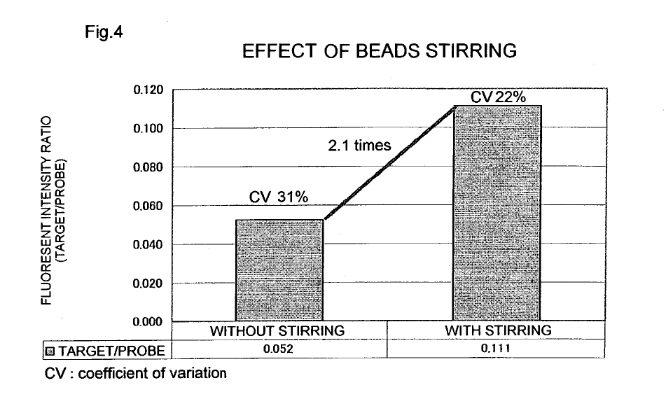

5. Scanner analysis (digitization) was conducted by known methods. The results are given in FIG. 4.

Components and Concentrations of Probes and Targets

1. Probes (Stamp Concentration) Cy3-gapdh in 1×PBS concentration 308 ng/microliter

TABLE 1
2. Targets

| | | | Without | |
|---|---|---|---|---|
| | Concentration | With Stirring | Stirring | Final Concentration |
| Cy5-dUTP- | 254 ng/microliter | 1.93 | 1.93 | 1.4 ng/microliter |
| gapdh | | | | |
| 20 × SSC | | 42.5 | 52.5 | 3 × SSC |
| yeast tRNA | 10 microgram | 35 | 35 | 1 microgram |
| | /microliter | | | /microliter |
| 10 × blocking | | 35 | 35 | 1 × b.s. |
| solution | | | | |
| 10% SDS | | 7 | 7 | 0.2% SDS |
| Beads | | 10 | — | |
| in 20 × SSC | | | | |
| DW | | 218.57 | 218.57 | |
| Total | | 350 microliters | 350 microliters | |

*Beads in 20 × SSC: Beads 0.05 g + 20 × SSC 1,000 microliters

FIG. 4 shows the ratio of the hybridized target fluorescent intensity to the probe fluorescent intensity when the target solution was stirred with beads and when it was not stirred during hybridization. When bead stirring was not conducted, the ratio was 0.052, and when bead stirring was conducted, the ratio was 0.111.

Compared to hybridization conducted without stirring (comparative example), the use of the magnetic bead stirring method (method of stirring a reaction solution, embodiment of the present invention) with a cDNA microarray yielded a more uniform hybrid signal with high sensitivity as a result of effective hybridization (the effect of stirring), even at identical concentrations of target DNA solution.

The method of the present invention permits the effective stirring of reaction solutions in micro reaction vessels in cases such as when hybridizing target DNA in DNA chips and DNA microarrays. In particular, when hybridizing target DNA in a DNA chip or DNA microarray, stable hybridization is achieved in a shorter period.

The present disclosure relates to the subject matter contained in Japanese Patent Application No. 2001-384916 filed on Dec. 18, 2001 and Japanese Patent Application No. 2002-339344 filed on Nov. 22, 2002, which is expressly incorporated herein by reference in its entirety.

What is claimed is:

1. A method of stirring a reaction solution in a micro reaction vessel by imparting magnetic field fluctuation from the exterior of said reaction vessel to magnetic beads contained in said reaction solution.

2. The method according to claim 1, wherein said magnetic field fluctuation is conducted by sequentially exciting multiple electromagnets or by displacing permanent magnets positioned outside the reaction vessel.

3. The method according to claim 1 or 2, wherein the micro reaction vessel is a DNA chip or DNA microarray hybridization vessel.

4. The method according to any of claims 1 to 3, wherein the thickness of the interior of the micro reaction vessel ranges from 0.1 to 1 mm and the diameter of the magnetic beads ranges from 0.1 to 20 percent of said thickness.

5. The method according to any of claims 1 to 4, wherein the volume of said micro reaction vessel ranges from 10 to 1,000 microliters.

6. The method according to any of claims 1 to 5, wherein magnetic beads are employed that constitute from 0.1 to 10 percent of the volume of the reaction solution.
